(19) United States
(12) Patent Application Publication
(10) Pub. No.: US 2022/0134051 A1
(43) Pub. Date: May 5, 2022

(54) MULTIMODAL SENSORY STIMULATION

(71) Applicant: University of Iowa Research Foundation, Iowa City, IA (US)

(72) Inventor: Aaron Boes, Iowa City, IA (US)

(21) Appl. No.: 17/518,301

(22) Filed: Nov. 3, 2021

Related U.S. Application Data
(60) Provisional application No. 63/109,163, filed on Nov. 3, 2020.

(51) Int. Cl. A61M 21/02; A61B 5/291

(52) U.S. Cl. CPC A61M 21/02; A61M 2021/0044; A61B 5/291

(57) ABSTRACT

A multimodal sensory device for promoting sleep in a subject, including a first sensory delivery component, a second sensory delivery component, and a controller module. The sensory device wherein the first and second sensory delivery components are constructed and arranged for delivering a first and second sensory stimuli to the subject and the controller is constructed and arranged to deliver the first and second sensory stimuli at a synchronized frequency. A method for promoting sleep in a subject including delivering to the subject at least one sensory stimuli at a frequency of between about 1 and 60 hz.

### CROSS-REFERENCE TO RELATED APPLICATION(S)

This application claims the benefit under 35 U.S.C. § 119(e) to U.S. Provisional Application Ser. No. 63/109,163, filed Nov. 3, 2020, and entitled “Multimodal Sensory Stimulation”, which is hereby incorporated herein by reference in its entirety for all purposes.

### GOVERNMENT SUPPORT

This invention was made with government support under RO1 NS114405 and R24 MH120441, awarded by the National Institutes of Health. The government has certain rights in the invention.

### BACKGROUND

Insomnia and related sleep disorders can have significant impact on quality of life and overall health. Pharmacologic intervention can be effective in the short term, but long term use frequently results in tolerance and/or dependence. There is a need for non-pharmacologic interventions to aid in sleep.

### BRIEF SUMMARY

Described herein are various embodiments relating to compositions of and methods for treating insomnia and/or promoting sleep.

Further disclosed herein are methods for promoting sleep in a subject by delivering to the subject at least one sensory stimulus at a frequency of between about 1 and 60 Hz. In certain aspects, the at least one stimulus is delivered at about 40 Hz. In certain embodiments, the at least one sensory stimulus is visual, tactile, or auditory. In certain implementations, the at least one sensory stimulus is a visual stimulus.

According to certain embodiments, the method further comprises at least a second sensory stimuli wherein the sensory modality of the second sensory stimulus is different from that of the first sensory stimulus.

According to certain further embodiments, the disclosed method further comprises concurrently presenting at least a third sensory stimulus wherein the sensory modality of the third sensory stimulus is different from that of the first or second sensory stimuli.

In certain aspects, the at least one sensory stimulus is delivered for a time interval of from about one to about 30 minutes. In certain implementations, the time interval is about from 5 minutes to about 10 minutes.

In certain embodiments, the intensity of the at least one sensory stimulus is adjustable by the subject. In further embodiments, where two or more stimuli are delivered, the intensity of each of the stimuli is independently adjustable by the subject.

In certain embodiments, the method further comprises placing at least one EEG electrode on the subject and measuring brain oscillations of the subject. In exemplary implementations of these embodiments, the one or more sensory stimuli are delivered in phase with the recorded brain oscillations of the subject.

Further disclosed herein is a multimodal sensory device for promoting sleep in a subject, comprising: a first sensory delivery component; a second sensory delivery component; and a controller module, wherein the first and second sensory delivery components are constructed and arranged for delivering a first and second sensory stimuli to the subject and the controller is constructed and arranged to deliver the first and second sensory stimuli at a synchronized frequency. In certain aspects, the first and second sensory stimuli are visual stimuli, auditory stimuli, or tactile stimuli. In certain embodiments, the first and second sensory stimuli are different. In certain implementations, the controller is constructed and arranged to deliver the synchronized frequency at between about 1 Hz and about 60 Hz.

Further disclosed herein is a multimodal sensory system for multimodal sensory stimulation of a subject, comprising: a first sensory delivery component configured to deliver a first stimuli to a subject; a second sensory delivery component configured to deliver a second stimuli to the subject; and a synchronizing controller configured to synchronize the delivery of the first and second stimuli, wherein the first and second stimuli are different and each is selected from the group consisting of visual, auditory and tactile stimuli. In certain aspects, the first and second stimuli are synchronized at between about 1 Hz and about 60 Hz.

According to certain aspects, the system further comprises an adjustment component configured to adjust the intensity of the first and/or second stimuli. In certain implementations, the stimulus intensity is adjustable by way of input from the subject.

In certain embodiments, the system also includes at least one EEG electrode configured for measuring brain oscillations of the subject. In exemplary implementations of these embodiments, the first and second stimuli are delivered in phase with recorded brain oscillations of the subject.

While multiple embodiments are disclosed, still other embodiments of the disclosure will become apparent to those skilled in the art from the following detailed description, which shows and describes illustrative embodiments of the disclosed apparatus, systems and methods. As will be realized, the disclosed apparatus, systems and methods are capable of modifications in various obvious aspects, all without departing from the spirit and scope of the disclosure. Accordingly, the drawings and detailed description are to be regarded as illustrative in nature and not restrictive.

### BRIEF DESCRIPTION OF THE DRAWING(S)

FIG. 1 is a schematic overview of the system, according to one implementation.

### DETAILED DESCRIPTION

Before the present methods, articles, systems, and/or devices, are disclosed and described, it is to be understood that they are not limited to specific method, article, system or device unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Ranges can be expressed herein as from “about” one particular value, and/or to “about” another particular value. When such a range is expressed, a further aspect includes from the one particular value and/or to the other particular value. Similarly, when values are expressed as approximations, by use of the antecedent “about,” it will be understood that the particular value forms a further aspect. It will be further understood that the endpoints of each of the ranges are significant both in relation to the other endpoint, and independently of the other endpoint. It is also understood that there are a number of values disclosed herein, and that each value is also herein disclosed as “about” that particular value in addition to the value itself. For example, if the value “10” is disclosed, then “about 10” is also disclosed. It is also understood that each unit between two particular units are also disclosed. For example, if 10 and 15 are disclosed, then 11, 12, 13, and 14 are also disclosed.

As used herein, the term “subject” refers to the target of administration, e.g. an animal. Thus the subject of the herein disclosed methods can be a human, non-human primate, horse, pig, rabbit, dog, sheep, goat, cow, cat, guinea pig or rodent. The term does not denote a particular age or sex. Thus, adult and newborn subjects, whether male or female, are intended to be covered. In one aspect, the subject is a mammal. A patient refers to a subject afflicted with a disease or disorder. The term “patient” includes human and veterinary subjects.

As used herein, the term “synergistic effect” or grammatical variations thereof means and includes a cooperative action encountered in a combination of two or more stimuli in which the sleep-promoting impact of the two or more stimuli exceeds the sum of the impact of each stimulus alone.

The term “synergistically effective amount,” as used herein, means and includes an amount (e.g. duration, frequency, and/or intensity) of two or more stimuli that provides a synergistic effect defined above.

Insomnia can be described as primary, secondary, or co-morbid. Primary insomnia involves sleep parameter deficiencies not attributable to a medical, environmental, or psychiatric cause. Secondary insomnia includes sleep parameter deficiencies that are associated with another condition. Co-morbid insomnia includes primary insomnia concomitant with one or more other conditions. Insomnia can be further characterized as transient, acute or short-term, and chronic. Transient insomnia refers to sleep parameter deficiencies lasting a few nights. Acute or short-term insomnia refers to sleep parameter deficiencies lasting less than a month. Chronic insomnia refers to sleep parameter deficiencies lasting at least one month.

As used herein, the term “treatment” refers to the medical management of a patient with the intent to cure, ameliorate, stabilize, or prevent a disease, pathological condition, or disorder. This term includes active treatment, that is, treatment directed specifically toward the improvement of a disease, pathological condition, or disorder, and also includes causal treatment, that is, treatment directed toward removal of the cause of the associated disease, pathological condition, or disorder. In addition, this term includes palliative treatment, that is, treatment designed for the relief of symptoms rather than the curing of the disease, pathological condition, or disorder; preventative treatment, that is, treatment directed to minimizing or partially or completely inhibiting the development of the associated disease, pathological condition, or disorder; and supportive treatment, that is, treatment employed to supplement another specific therapy directed toward the improvement of the associated disease, pathological condition, or disorder. In various aspects, the term covers any treatment of a subject, including a mammal (e.g., a human), and includes: (i) preventing the disease from occurring in a subject that can be predisposed to the disease but has not yet been diagnosed as having it; (ii) inhibiting the disease, i.e., arresting its development; or (iii) relieving the disease, i.e., causing regression of the disease. In one aspect, the subject is a mammal such as a primate, and, in a further aspect, the subject is a human. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, etc.).

As used herein, the term “prevent” or “preventing” refers to precluding, averting, obviating, forestalling, stopping, or hindering something from happening, especially by advance action. It is understood that where reduce, inhibit or prevent are used herein, unless specifically indicated otherwise, the use of the other two words is also expressly disclosed.

Disclosed herein are systems and methods for promoting for promoting sleep in subjects in need thereof. In certain aspects, the disclosed methods comprise delivering to the subject at least one sensory stimuli at a predetermined frequency. In certain embodiments, the at least one sensory stimuli is delivered at a frequency of from about 1 Hz to about 60 Hz. In exemplary implementations, the frequency is delivered at a frequency of about 40 Hz.

In certain aspects, the at least one sensory stimuli is visual, tactile, or auditory. In exemplary implementations the at least one sensory stimuli is visual. In certain embodiments, the amplitude of the one or more stimuli is adjustable by the subject (e.g., the subject can adjust the brightness, volume or tactile intensity to a level that is comfortable to the subject).

In certain embodiments, the one or more stimuli is delivered for from about 1 minute to about 30 minutes. In exemplary implementations, the one or more stimuli is delivered for from about 5 minutes to about 10 minutes.

According to certain further embodiments, the disclosed method further comprises concurrently presenting at least a second sensory stimuli wherein the sensory modality of the second sensory stimulus is different from that of the first sensory stimuli. In exemplary implementations, the first sensory stimulus is visual, and the second sensory stimuli is auditory. In certain alternative implementations, the first sensory stimulus is visual, and the second sensory stimuli is tactile. In further alternative implementations, the first sensory stimulus is auditory, and the second sensory stimuli is tactile. In exemplary implementations, the first and second stimuli are presented at a frequency with one another.

In certain further embodiments, the disclosed method further comprises presenting at least a third sensory stimuli wherein the sensory modality of the third sensory stimuli is different from that of the first or second sensory stimuli. In exemplary implementations, the frequencies of the three sensory stimuli are each in phase with one another.

In certain implementations, the delivery of two or more stimuli provides a synergistic effect in promoting sleep in the subject, relative to the delivery of any single stimulus alone. In exemplary implementations, the two or more stimuli are delivered at a synergistically effective amount.

In certain embodiments, the subject has been diagnosed with insomnia. In certain embodiments, the subject has primary insomnia. In alternative embodiments, the subject has secondary insomnia. In further embodiments, the subject suffers from transient insomnia, diagnosed or undiagnosed.

According to still further embodiments, the disclosed method further comprises placing at least one EEG electrode on the subject and measuring brain oscillations of the subject. In exemplary implementations of these embodiments, the one or more sensory stimuli are delivered in phase with frequency recorded through the at least one EEG electrode. According to further embodiments, a two lead EEG system is utilized. In exemplary implementations of these embodiments, the two electrodes are configured in a closed loop arrangement and stimulation is synchronized to ongoing brain oscillations. Such stimulation has the effect of entraining brain oscillations and driving them into a lower frequency range, which tends to be seen in association with sleepiness.

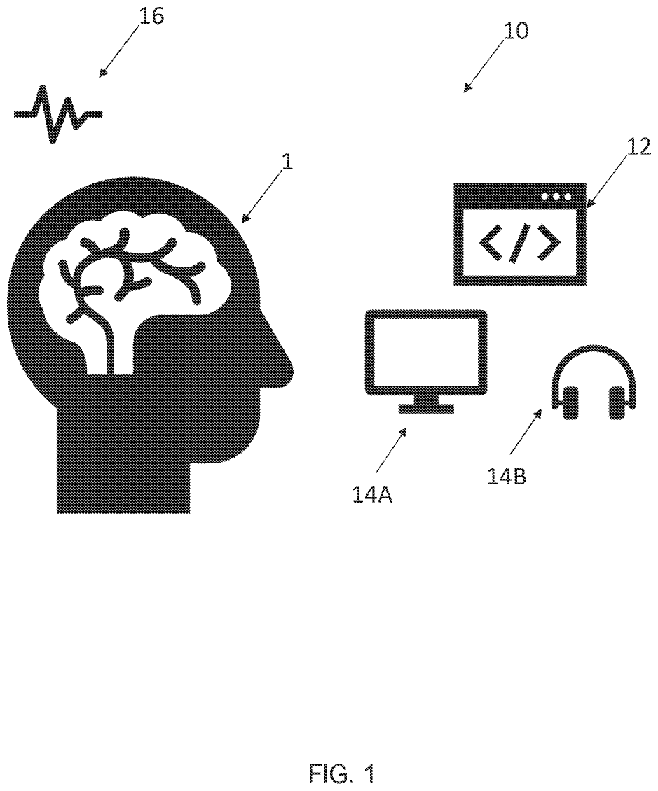

Turning to the drawings in greater detail, FIG. 1 depicts an exemplary implantation of the system 10, according to certain implementations. In these implementations of the system 10, a controller module 12 is provided that is in operational communication with one or more sensory delivery components 14A, 14B for the application of the various modes of sensory stimuli to the subject 1, as would be readily appreciated.

It is further appreciated that the various sensory delivery component 14A, 14B can be in physical communication and/or electrical communication with one another, such as a visual sensory delivery component 14A in electronic communication with an auditory sensory delivery component 14B, such as via a wired or wireless connection (not shown). One of skill in the art would appreciate such configurations for the various modes of sensory stimuli presented herein.

In various implementations of the system 10, the first 14A and second 14B sensory delivery components are constructed and arranged for delivering a first and second sensory stimuli to the subject and the controller 12 is constructed and arranged to deliver the first and second sensory stimuli at a synchronized frequency.

In certain implementations, the subject 1 is thus can be delivered photic-auditory stimulation via the sensory delivery components 14A, 14B as commanded by the controller module 12 according to a series of commands or other steps to effectuate the synchronous application of stimuli to the subject 1. In certain implementations, the first and second stimuli are different and each is selected from the group consisting of visual, auditory and tactile stimuli.

In certain implementations, and as shown in FIG. 1, the system also comprises at least one EEG electrode 16 configured for measuring brain oscillations of the subject 1 while stimulus is being applied.

### EXAMPLES

The following examples are put forth so as to provide those of ordinary skill in the art with a complete disclosure and description of how the compounds, compositions, articles, devices and/or methods claimed herein are made and evaluated, and are intended to be purely exemplary of the invention and are not intended to limit the scope of what the inventors regard as their invention. However, those of skill in the art should, in light of the present disclosure, appreciate that many changes can be made in the specific embodiments which are disclosed and still obtain a like or similar result without departing from the spirit and scope of the invention.

40 Hz photic-auditory stimulation was delivered in neurosurgery patients in 1 minute blocks on 4 different subject. Two of the subjects appeared to get sleepy during the stimulation protocol, including another family member in the room for one experiment.

Although the disclosure has been described with reference to preferred embodiments, persons skilled in the art will recognize that changes may be made in form and detail without departing from the spirit and scope of the disclosed apparatus, systems and methods.

What is claimed is:

1. A method for promoting sleep in a subject comprising delivering to the subject at least one sensory stimulus at a frequency of between about 1 and 60 Hz.

2. The method of claim 1, wherein the at least one stimulus is delivered at about 40 Hz.

3. The method of claim 1, wherein the at least one sensory stimulus is visual, tactile, or auditory.

4. The method of claim 1, wherein the at least one sensory stimulus is a visual stimulus.

5. The method of claim 2, further comprising concurrently presenting at least a second sensory stimulus wherein the sensory modality of the second sensory stimulus is different from that of the first sensory stimulus.

6. The method of claim 5, further comprising concurrently presenting at least a third sensory stimulus wherein the sensory modality of the third sensory stimulus is different from that of the first or second sensory stimuli.

7. The method of claim 6, wherein the stimuli are delivered for a time interval of from about one to about 30 minutes.

8. The method of claim 7, wherein the time interval is about from 5 minutes to about 10 minutes.

9. The method of claim 1, wherein the intensity of the at least one sensory stimulus is adjustable by the subject.

10. The method of claim 1, further comprising placing at least one EEG electrode on the subject and measuring brain oscillations of the subject.

11. The method of claim 10, wherein the one or more sensory stimulus is delivered in phase with the recorded brain oscillations of the subject.

12. A multimodal sensory device for promoting sleep in a subject, comprising:
a) a first sensory delivery component;
b) a second sensory delivery component; and
c) a controller module,
wherein the first and second sensory delivery components are constructed and arranged for delivering a first and second sensory stimuli to the subject and the controller is constructed and arranged to deliver the first and second sensory stimuli at a synchronized frequency.

13. The device of claim 12, wherein the first and second sensory stimuli are visual stimuli, auditory stimuli, or tactile stimuli.

14. The device of claim 13, wherein the first and second sensory stimuli are different.

15. The device of claim 12, wherein the controller is constructed and arranged to deliver the synchronized frequency at between about 1 Hz and about 60 Hz.

16. A multimodal sensory system for multimodal sensory stimulation of a subject, comprising:
a) a first sensory delivery component configured to deliver a first stimuli to a subject;
b) a second sensory delivery component configured to deliver a second stimuli to the subject; and
c) a synchronizing controller configured to synchronize the delivery of the first and second stimuli,
wherein the first and second stimuli are different and each is selected from the group consisting of visual, auditory and tactile stimuli.

17. The system of claim 16, wherein the first and second stimuli are synchronized at between about 1 Hz and about 60 Hz.

18. The system of claim 16, further comprising an adjustment component configured to adjust the intensity of the first and/or second stimuli via subject input.

19. The system of claim 16, further comprising at least one EEG electrode configured for measuring brain oscillations of the subject.

20. The system of claim 19, wherein the first and second stimuli are delivered in phase with recorded brain oscillations of the subject.
